(19) United States
(12) Patent Application Publication
(10) Pub. No.: US 2006/0189693 A1
(43) Pub. Date: Aug. 24, 2006

(54) Methods of treatment with lxr agonists

(76) Inventors: Frank C. Barone, King of Prussia, PA (US); Robert W. Coatney, King of Prussia, PA (US); Jeffrey J. Legos, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/565,495

(22) PCT Filed: Jul. 22, 2004
(86) PCT No.: PCT/US04/23658

§ 371 Date: Jan. 20, 2006

Related U.S. Application Data
(60) Provisional application No. 60/489,202, filed on Jul. 22, 2003.

(51) Int. Cl. A61K 31/195

(52) U.S. Cl. 514/567; 562/442

(57) ABSTRACT

The present invention relates generally to the use of LXR agonists in the prevention and/or treatment of cardiovascular pathology.

### FIELD OF THE INVENTION

The present invention relates generally to the use of LXR agonists in the prevention and/or treatment of cardiovascular pathology.

### BACKGROUND OF THE INVENTION

LXRα and LXRβ (collectively LXR) are nuclear hormone receptors that regulate the metabolism of several important lipids, including cholesterol (Peet, et al. Curr Opin Genet Dev, 1998; 8(5):571-5). The nucleotide and amino acid sequences of LXRα are shown in FIGS. 1 and 2 (SEQ ID NOs:1 and 2), respectively. The nucleotide and amino acid sequences of LXRβ are shown in FIGS. 3 and 4 (SEQ ID NOs:3 and 4), respectively. The LXRs regulate the expression of target genes by binding to short stretches of DNA, termed LXR response elements (LXREs), as heterodimers with the retinoid X receptors (RXR) (Apfel, et al., Mol Cell Biol, 1994; 14(10):7025-35; Teboul, et al., Proc Natl Acad Sci USA, 1995; 92(6):2096-100; Song, et al., Proc Natl Acad Sci USA, 1994; 91(23):10809-13; and Willy, et al., Genes Dev, 1995; 9(9):1033-45). LXREs have been identified in the regulatory regions of a number of genes involved in cholesterol homeostasis including CYP7A1 (Peet, et al., Cell, 1998; 93(5):693-704), which catalyses the first and rate-limiting step in bile acid biosynthesis, the cholesterol ester transport protein (Lu, et al, J Clin Invest, 2000; 105(4):513-20), the transcription factor SREBP-1C (Repa, et al., Genes Dev, 2000; 14(22):2819-30 and Schultz, et al, Genes Dev, 2000; 14(22):2831-8.), and apolipoprotein E (apoE)(Laffitte, et al., Proc Natl Acad Sci USA, 2001; 98(2):507-12). LXREs have also been identified in the genes encoding the ATP binding cassette transporters (ABC) A1 and Gl (Costet, et al., J Biol Chem, 2000; 275(36):28240-5; Repa, et al., Science, 2000; 289(5484): 1524-9; Venkateswaran, et al., J Biol Chem, 2000; 275(19):14700-7; Venkateswaran, et al., Proc Natl Acad Sci USA, 2000; 97(22): 12097-102; Schwartz, et al., Biochem Biophys Res Commun, 2000; 274(3):794-802; and Re pa, et al, Annu Rev Cell Dev Biol 16:459-481), which mediate the efflux of phospholipids and cholesterol from macrophages, intestinal enterocytes and other cell types.

Currently, patients with elevated levels of cholesterol are treated using the compounds that inhibit the body's endogenous cholesterol synthesis. As important components of the complex system that regulates cholesterol levels in the body, the LXRs have also been proposed as targets for the prophylaxis and treatment of hypercholesteraemia (raised levels of plasma cholesterol) and its associated atherosclerotic diseases.

Cardiac hypertrophy is an increase in myocardial (heart) muscle mass where wall thickness increases in size because the heart has to work harder to maintain normal physiologic function. Cardiac hypertrophy may be caused by both hemodynamic stresses and non-hemodynamic factors. Included in the hemodynamic stresses that contribute to increased wall thickness (e.g., cardiac hypertrophy) are pressure overload from hypertension and/or arteriosclerosis or volume overload from sodium and water retention. Non-hemodynamic factors that contribute to developing pathology may include activation of the renin-angiotensin-aldosterone system (which also increases volume and pressure overload) and the level of fibrosis or stiffness in the myocardium. These events may lead to increased wall thickness and decreased ventricular chamber diameter.

This increase in wall thickness places patients at risk for developing cardiovascular pathology which may include coronary heart disease, heart failure, congestive heart failure (herein “CHF”), myocardial infarction, as well as other cardiovascular complications which are associated with a significant increase in mortality. Reducing or reversing cardiac hypertrophy to levels that are approaching that of healthy patients has been associated with reduced arrhythmias, improved cardiac function, and reduced risk of heart failure including congestive heart failure and an improvement in coronary blood flow reserves enabling patients to live healthier, longer lives.

The use of agonists of LXR and their pharmaceutical formulations to reverse cholesterol transport and treat atherosclerotic cardiovascular diseases have been reported. The present inventors have now discovered that LXR agonists have a property of reducing cardiac hypertrophy in mammals suffering from cardiac hypertrophy.

### SUMMARY OF THE INVENTION

In one aspect, the present invention provides a method of treating or preventing cardiovascular pathology, including, but not limited to cardiac hypertrophy, coronary heart disease, arrhythmia, restricted coronary blood flow, arteriosclerosis, heart failure, congestive heart failure (CHF), and myocardial infarction; comprising, administering a therapeutically effective amount of LXR agonist, or a pharmaceutically acceptable salt, solvate, or physiologically functional derivative thereof.

In a further aspect, the invention also relates to a pharmaceutical composition comprising a therapeutically effective amount of LXR agonist, or a pharmaceutically acceptable salt, solvate, or physiologically functional derivative thereof, and a pharmaceutically acceptable carrier for the treatment or prevention of cardiovascular pathology, including, but not limited to cardiac hypertrophy, coronary heart disease, arrhythmia, restricted coronary blood flow, arteriosclerosis, heart failure, congestive heart failure (CHF), and myocardial infarction.

Yet in a further aspect, the present invention relates to the use of a LXR agonist in the preparation of a medicament for the treatment or prevention of cardiovascular pathology, including, but not limited to cardiac hypertrophy, coronary heart disease, arrhythmia, restricted coronary blood flow, arteriosclerosis, heart failure, congestive heart failure (CHF), and myocardial infarction.

### BRIEF DESCRIPTION OF THE DRAWINGS

FIG. 1 shows the nucleotide sequence of human LXRα (SEQ ID NO:1) from Genebank, accession NM_{—}005693.

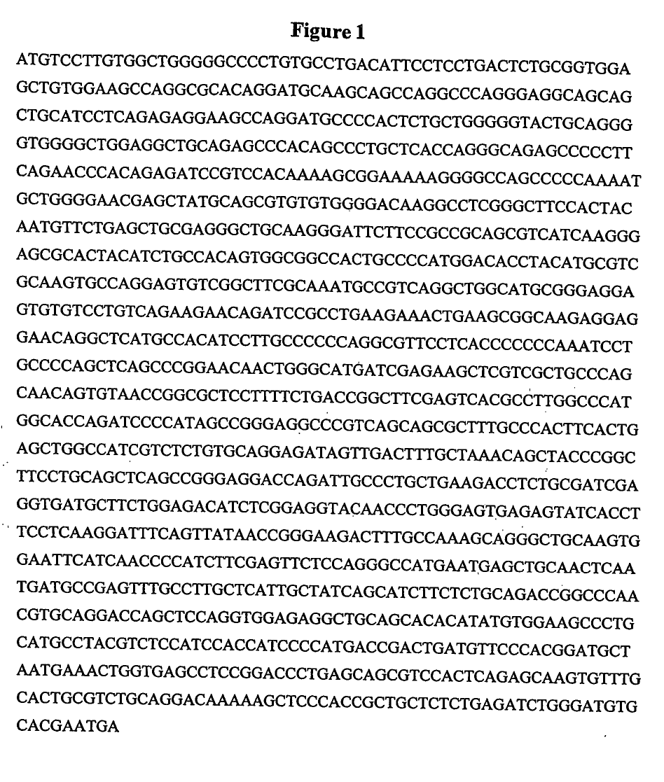

FIG. 2 shows the deduced amino acid sequence of human LXRα (SEQ ID NO:2) from Genebank accession NP_{—}005684.

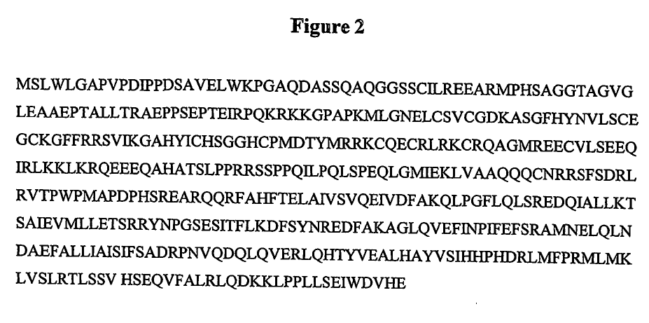

FIG. 3 shows the nucleotide sequence of human LXRβ from Genebank accession (SEQ ID NO:3) from Genbank accession XM_{—}046419.

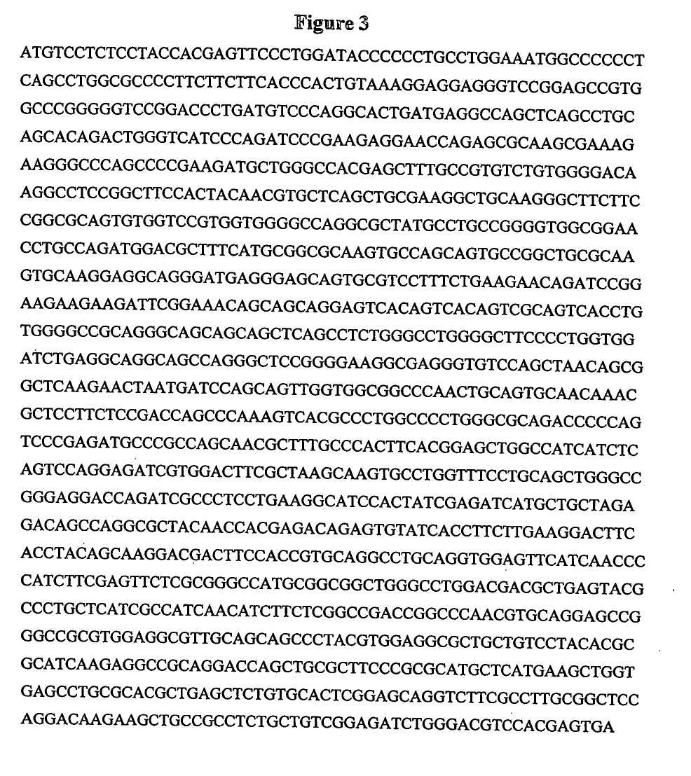

FIG. 4 shows the deduced amino acid sequence of human LXRβ (SEQ ID NO:4) from Genebank accession XP_{—}046419.

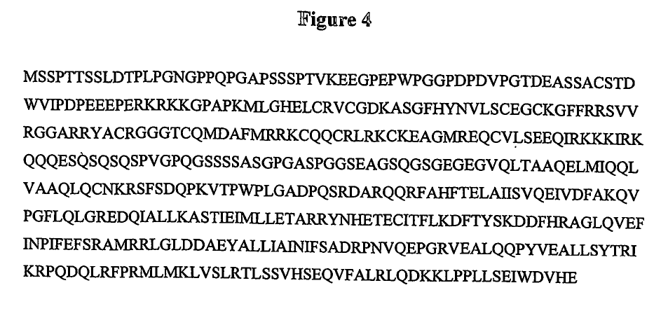

### DETAILED DESCRIPTION OF THE INVENTION

In view of an unmet medical need of providing new treatments for disorders associated with cardiovascular pathology, a study was commenced that investigated inhibitors having a property of reducing cardiac hypertrophy. The present invention was based, in part, on that study.

The instant invention provides a method for reducing cardiovascular pathology in a mammal suffering from cardiovascular pathology, comprising administering an effective amount of a LXR agonist to reduce said cardiovascular pathology.

As used herein, “reduce” or “reducing” refers to a decrease in the severity of or cessation of a cardiovascular pathology. The severity of cardiovascular pathology may be decreased by reducing the left ventricular muscle mass of an animal to any extent, including but not limited to reducing left ventricular muscle mass equivalent to that observed at baseline or to left ventricular muscle mass considered to be within normal left ventricular mass for a healthy animal of the same species and having similar physical characteristics.

As used herein, “baseline left ventricular mass” refers to left ventricular muscle mass of an animal prior to receiving any therapeutic method or compound of the invention.

As used herein, “normal left ventricular mass” refers to: (1) the amount of left ventricular mass in a healthy animal of the same species and having similar physical characteristics including but not limited to gender, age, weight, height, blood pressure, and underlying disease, and/or (2) the amount of left ventricular mass in a animal of the same species and having similar physical characteristics including but not limited to gender, age, weight, height, blood pressure, and underlying disease prior to treatment using a method or compound of the invention.

A “healthy animal” as used herein refers to an animal that does not show thickening of the left ventricular wall above normal. Healthy animals may not show signs or symptoms of cardiovascular pathology, and may be free from any other underlying disease or gross morbidity.

As used herein, “cardiovascular pathology” refers to a cardiovascular complication or risk thereof and may include but is not limited to cardiac hypertrophy, coronary heart disease, arrhythmia, restricted coronary blood flow, arteriosclerosis, heart failure, congestive heart failure (CHF), myocardial infarction, as well as others.

As used herein, “cardiac hypertrophy” refers to increased left ventricular mass above normal. Cardiac hypertrophy may manifest as heart wall growth that causes a narrowing of the ventricular chambers.

As used herein, “increased left ventricular muscle mass” refers to growth or thickening of the left ventricular wall upon the onset of one or more cardiovascular pathology.

As used herein “hemodynamic stress” refers to any factors contributing to pressure, viscosity and/or volume overload of the cardiac system. Hemodynamic stress that may contribute to pressure overload may include but is not limited to hypertension/arteriosclerosis. Hemodynamic stress that may contribute to volume overload may include but is not limited to sodium and water retention. Hemodynamic stress contributes to increased left ventricular wall thickness (e.g., cardiac hypertrophy).

As used herein “non-hemodynamic factors” refers to any factors contributing to pressure and/or volume overload of the cardiac system. Non-hemodynamic factors may include but are not limited to activation of the renin-angiotensin-aldosterone system, and increased fibrosis or stiffness in the myocardium.

The term “LXR agonist” means any compound that enhances the biological activities of LXRα and/or LXRβ. LXR agonists are well known. LXR agonists of the present invention may be selected from compounds of formulas (I), (II), (III), (IV), and (V). The compounds of formulas (I), (II), (III), (IV), and (V) are described in more detail below. Other examples of LXR agonists which form part of instant invention are described in:

WO2002090375 published Nov. 14, 2002;

WO2002058532 published Aug. 1, 2002;

WO200211708 published Feb. 14, 2002;

WO200160818 published Aug. 23, 2001;

WO200115676 published Mar. 8, 2001;

WO200103705 published Jan. 18, 2001; and

WO2000666111 published Nov. 9, 2000.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. The solvent used may be a pharmaceutically acceptable solvent. Examples of suitable pharmaceutically acceptable solvents include, without limitation, water, ethanol and acetic acid.

International Patent Application WO 00/54759 (Tularik Inc. US) discloses compounds of formula (I): 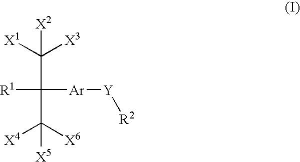
wherein:

Ar represents an aryl group; R¹ is —

OH, —O—(C₁-C₇)alkyl, —OC(O)—(C₁-C₇)alkyl, —O—(C₁-C₇)heteroalkyl, —OC(O)—(C₁-C₇)heteroal kyl, —CO₂H, —NH₂, —NH(C₁-C₇)alkyl, —N((C₇-C₇)alkyl)₂ or —NH—S(O)₂—(C₁-C₅)alkyl;

R² is (C₁-C₇)alkyl, (C₁-C₇)heteroalkyl, aryl and aryl(C₁-C₇)alkyl;

X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and

Y is —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— or —N(R¹²)C(O)O—, wherein R12 and R13 are each independently hydrogen, (C₁-C₇)aryl, (C₁-C₇)heteroalkyl, aryl and aryl(C₁-C₇)alkyl, and optionally when Y is —N(R¹²)S(O)ₘ— or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2, as being useful as agonists of LXR and their use in pharmaceutical formulations to reverse cholesterol transport and treat atherosclerotic cardiovascular diseases and related diseases.

With respect to the compounds of formula (I), the term “alkyl”, by itself or as part of another substituent, means, unless otherwise stated, a straight or branched chain, or cyclic hydrocarbon radical, or combination thereof, which may be fully saturated, mono- or polyunsaturated and may include di- and multi-radicals, having the number of carbons designated (i.e., C₁₋₁₀ means one to ten carbons). Examples of saturated hydrocarbon radicals include groups such as methyl, ethyl, n-propyl, isopropyl, n-butyl, t-butyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl”, unless otherwise noted, is also meant to include those derivatives of alkyl defined in more detail below as “cycloalkyl” and “alkylene.” The term “alkylene” by itself or as part of another substituent means a divalent radical derived from alkane, as exemplified by —CH₂CH₂CH₂CH₂—. An alkyl group may have from 1 to 24 carbon atoms. A “lower alkyl” or “lower alkylene” is a shorter chain alkyl or alkylene group, generally having eight, fewer than eight, four or fewer carbon atoms.

The term “alkoxy,” employed alone or in combination with other terms means, unless otherwise stated, an alkyl group, as defined above, connected to the remainder of the molecule via an oxygen atom, such as, for example, methoxy, ethoxy, 1-propoxy, 2-propoxy, and the higher homologs and isomers.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si, S, and wherein the nitrogen and sulfur atoms may optionally be oxidized and the nitrogen heteroatom may optionally be quarternized. The heteroatom(s) O, N and S may be placed at any position of the heteroalkyl group except for the position at which the alkyl group, is attached to the remainder of the molecule. Examples include —CH₂—CH₂—O—CH₃, —CH₂—CH₂—NH—CH₃, —CH₂—CH₂—N(CH₃), —CH₂—S—CH₂—CH₃, —CH₂—CH₂—S(O)—CH₃, —CH₂—CH₂—S(O)₂—CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene by itself or as part of another substituent means a divalent radical derived from heteroalkyl, as exemplified by —CH₂—CH₂—S—CH₂—CH₂— and —CH₂—S—CH₂—CH₂—NH—CH₂—. For heteroalkylene groups, heteroatoms may also occupy either or both of the chain termini. Still further, for alkylene and heteroalkylene linking groups, as well as all other linking groups described herein, no specific orientation of the linking group is implied.

The terms “cycloalkyl” and “heterocycloalkyl,” by themselves or in combination with other terms, represent, unless otherwise stated, cyclic versions of “alkyl” and “heteroalky” respectively. The terms “cycloalkyl” and “heterocycloalkyl” are also meant to include bicyclic, tricyclic and polycyclic versions thereof. Additionally, for heterocycloalkyl, a heteroatom may occupy the position at which the heterocyclyl is attached to the remainder of the molecule. Examples of cycloalkyl include cyclopentyl, cyclohexyl, 1-cyclohexyl, 3-cyclohexyl, cyclopentyl, bicyclo[2.2.1]heptyl, bicyclo[2.2.2]octyl, adamantyl, and the like. Example of heterocycloalkyl include 1-(1,2,5,6-tetrahydropyridyl), 1-piperidinyl, 2-piperidinyl, 3-piperidinyl, 4-morpholinyl, 3-morpholinyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen” by themselves or as part of another substituent, mean, unless otherwise stated, a fluorine, chlorine, bromine or iodine atom. Additionally, terms such as “fluoroalkyl”, are meant to include monofluoroalkyl and polyfluoroalkyl.

The term “aryl”, employed alone or in combination with other terms (e.g., aryloxy, arylthioxy, arylalkyl) means, unless otherwise stated, an aromatic substituent which may be a single ring or multiple rings (up to three rings) which are fused together or linked covalently. The rings may each contain from zero to four heteroatoms selected from N, O and S, wherein the nitrogen and sulfur atoms are optionally oxidized, and the nitrogen atom(s) are optionally quaternized. The aryl groups that contain heteroatoms may be referred to as “heteroaryl” and may be attached to the remainder of the molecule through a carbon atom or a heteroatom. Non-limiting examples of aryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidinyl, 4-pyrimidinyl, 5-benzothiazolyl, purinyl, 2-benzimidazolyl, 5-indolyl, 1-isoquinolinyl, 5-isoquinolinyl, 2-quinoxalinyl, 5-quinoxalinyl, 3-quinolinyl, and 6-quinolinyl. Substituents for each of the above noted aryl ring systems are selected form the group of acceptable substituents described below.

The terms “arylalkyl” and “arylheteroalkyl” are meant to include those radicals in which an aryl group is attached to an aryl group (e.g., benzyl, phenethyl, pyridylmethyl and the like) or a heteroalkyl group (e.g. phenoxymethyl, 2-pyridyloxymethyl, 1-napthyloxy-3-propyl, and the like). The arylaklyl and arylheteroalkyl groups may contain from 1 to 3 aryl moieties attached to the alkyl or heteroalkyl portion by a covalent bond or by fusing the ring to, for example, a cycloalkyl or heterocycloalkyl group. For arylheteroalkyl groups, a heteroatom may occupy the position at which the group is attached to the remainder of the molecule. For example, the term “arylheteroalkyl” is meant to include benzyloxy, 2-phenylethoxy, phenethylamine, and the like.

Each of the above terms (e.g., “alkyl”, “heteroalkyl”, “aryl” etc) is meant to include both substituted and unsubstituted forms of the indicated radical. Examples of substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and hetercycloalkenyl) may be a variety of groups selected from: —OR, ═O, ═NR′, N—OR′,

NR′R″, —SR′, -halogen, —SiR′R″R′″, —OC(O)R′, —CO₂R′, —CONR′R″,

OC(O)NR′R″, —NR″C(O)R′, —NR″C(O)NR′R′″, —NR″C(O)₂R′,

NHC(NH₂)═NH, —NR′C(NH₂)═NH, —NH—, C(NH₂)═NR′,

S(O)R′, —S(O)₂R′, —S(O)₂NR′R″, —CN and —NO₂ in a number ranging from zero to (2N+1), where N is the total number of carbon atoms in such a radical. Substituted alkyl groups may have from one to six independently selected substituents, more preferably from one to four independently selected substituents, most preferably from one to three independently selected substituents. In the substituents listed above, R′, R″ and R′″ each independently refer to hydrogen, unsubstituted (C₁₋₈)alkyl and heteroalkyl, unsubstituted aryl, aryl substituted with 1-3 halogens, unsubstituted alkyl, alkoxy or thioalkoxy groups or aryl-(C₁₋₄)alkyl groups.

When R′ and R″ are attached to the same nitrogen atom, they may be combined with the nitrogen atom to form a 5-, 6-, or 7-membered ring. For example, —NR′R″ is meant to include 1-pyrrolidinyl and 4-morpholinyl.

Similarly, substituents for the aryl groups are varied and selected from: -halogen, —OR′, —OC(O)R′, —NR′R″, —SR′, —R′, —CN, —NO₂, —CO₂R′, —CONR′R″, —OC(O)N R′R″, —NR″C(O)R′, —NR″C(O)₂R′, —NR″C(O)NR′R′″, —NH—C(NH₂)═NH, —NR′C(NH₂)═NH, —N H—C(NH₂)═NR′, —SOR′, —S(O)₂R′, —S(O)₂NR′R″, —N₃, —CH(Ph)₂, perfluor(C₁₋₄)alkoxy, and perfluoro(C₁₋₄)alkyl, in a number ranging from zero to the total number of open valences on the aromatic ring system; and where R′ and R″ are independently selected from hydrogen, (C₁₋₈)alkyl and heteroalkyl, unsubstituted aryl, (unsubstituted aryl)(C₁₋₄)alkyl, and (unsubstituted aryl)oxy-(C₁₋₄)alkyl. Substituted aryl groups may have from one to four independently selected substituents, more preferably from one to three independently selected substituents including from one to two independently selected substituents.

Two of the substituents on adjacent atoms of the aryl ring may optionally be replaced with a substituent of the formula -T-C(O)—(CH2)_{q}-U—, wherein T and U are independently —NH—, —O—, CH2 or a single bond, and q is an integer of from 0 to 2. Alternatively, two of the substituents on adjacent atoms of the aryl ring may optionally be replaced with a substituent of formula -A-(CH2)r-B—, wherein A and B are independently —CH2—, —O—, —NH—, S—, —S(O)—, —S(O)2-, —S(O)2NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —CH2)s-X—(CH2)t-, where s and t are integers of from 0 to 3, and X is —O—, —NR′—, —S—, —S(O)—, —S(O)2-, or —S(O)₂NR′—. The substituent R′ in —NR′— and S(O)2NR′— selected from hydrogen or unsubstituted (C1-6)alkyl.

The term “heteroatom” is meant to include oxygen (O), nitrogen (N), sulfur (S) and silicon (Si).

Compounds of formula (I) may be prepared using readily available starting materials or known intermediates. WO 00/54759 describes a number of possible synthetic routes for the production of such compounds, such as those depicted in scheme 1.

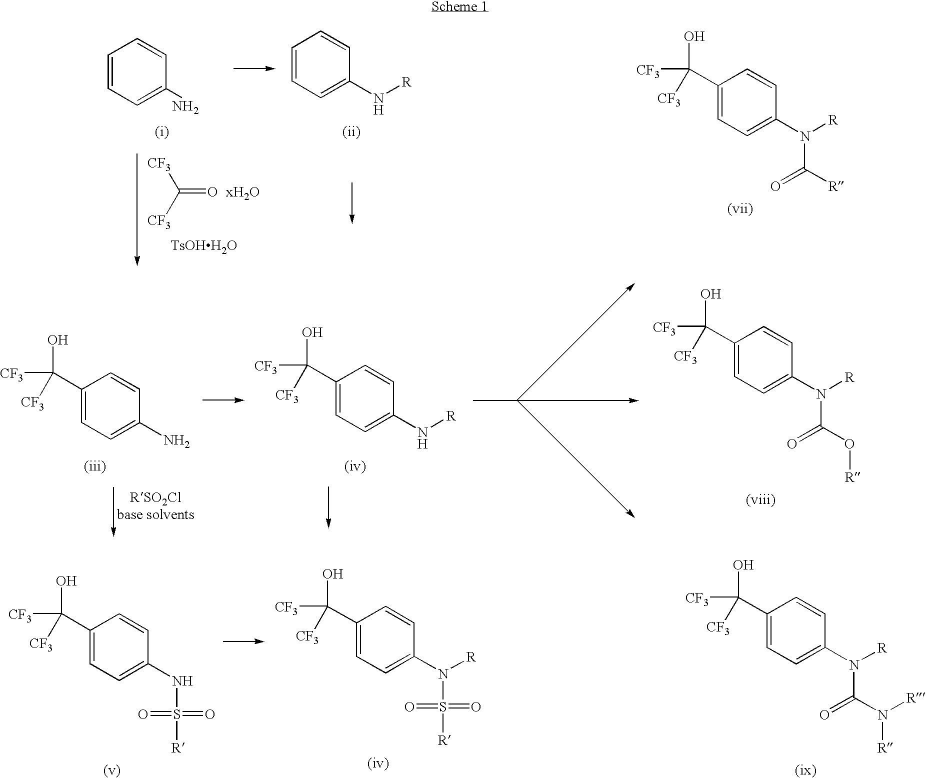

As shown in Scheme 1, aniline (i) (as representative of substituted anilines and other arylamines) may be alkylated, acylated or arylated (general addition of R group) to form (ii), or the aromatic ring may be derivatized with, for example, hexafluoroacetone to form (iii). Treatment of (iii) with an appropriate alkylating group, acylating group or arylating group provides (iv), which may be sulfonylated with, for example, an appropriate sulfonyl halide to form (vi). Alternatively, the aniline derivative may be sufonylated to form (v), which may then be alkylated or acylated to form compounds of formula (vi).

Other compounds of formula (I) may be formed by treating the substituted aniline (iv) (or iii), with reagents suitable for the formation of amides (vii), carbamates (viii) and ureas (ix). Various reagents are useful in the above scheme and may be found in, for example March, Advanced Organic Chemistry 4th ed. John Wiley & Sons, New York N.Y. (1992)

International Patent Application PCT/US01/27622 (SmithKline Beecham plc) discloses compounds of formula (II): 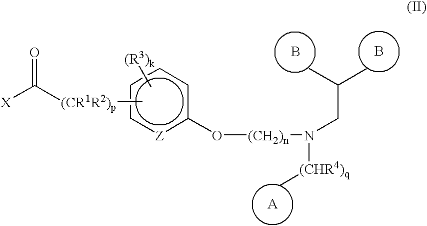
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 0-4;
when Z is N, k is 0-3;
each R³ is the same or different and is independently selected from the group consisting of halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
   a is 0, 1 or 2;
   R⁶ is selected from the group consisting of H, C₁₋₈alkyl, C₁₋₈alkoxy and C₂₋₈alkenyl;
   each R⁷ and R⁸ are the same or different and are each independently selected from the group consisting of H, C₁₋₈alkyl, C₂₋₈alkenyl, C₃₋₈alkynyl;
   R⁹ is selected from the group consisting of H, C₁₋₈alkyl and —NR⁷R⁸;
   R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₃₋₈cycloalkyl, aryl, 4-8 membered heterocycle, and 5-6 membered heteroaryl;
each ring B is the same or different and is independently selected from the group consisting of C₃₋₈cycloalkyl and aryl,
   as being useful as agonists of LXR and their use in pharmaceutical formulations to reverse cholesterol transport and treat atherosclerotic cardiovascular diseases and related diseases.

With respect to compounds of formula (II) the term “alkyl” refers to aliphatic straight or branched saturated hydrocarbon chains containing the specified number of carbon atoms. Examples of “alkyl” groups as used herein include but are not limited to methyl, ethyl, n-propyl, isopropyl, n-butyl, isobutyl, t-butyl, pentyl, hexyl, octyl and the like. The term “alkyl” also refers to substituted alkyl wherein the substituents are selected from the group consisting of halo, —OR⁷ and —SR⁷, where R⁷ is H or C₁₋₈alkyl. This definition of “alkyl” is also applicable to terms such as “thioalkyl” which incorporate the “alkyl” term. Thus, a “thioalkyl” as used herein refers to the group S—Ra where Ra is “alkyl” as defined.

The term “halo” refers to any halogen atom ie., fluorine, chlorine, bromine or iodine.

The term “alkenyl” refers to an aliphatic straight or branched unsaturated hydrocarbon chain containing at least one and up to three carbon-carbon double bonds. Examples of “alkenyl” groups as used herein include, but are not limited to, ethenyl and propenyl. The term “alkenyl” also refers to substituted alkenyl wherein the substituents are selected from the group consisting of halo, —OR7 and —SR7, where R7 is H or C1-8alkyl.

The term “alkoxy” refers to a group O—Ra where Ra is “alkyl” as defined above.

The term “alkenyloxy” refers to a group O—Rb where Rb is “alkenyl” as defined above.

The term “cycloalkyl” refers to a non-aromatic carbocyclic ring having the specified number of carbon atoms and up to three carbon-carbon double bonds. “Cycloalkyl” includes by way of example cyclopropyl, cyclobutyl, cyclopentyl, cyclohexyl, cycloheptyl, cyclooctyl, cyclobutenyl, cyclopentenyl, cyclohexenyl and bicyclic cycloalkyl groups such as bicycloheptane and bicyclo(2.2.1)heptene. The term “cycloalkyl” also refers to substituted cycloalkyl wherein the ring bears one or more substituents selected from the group consisting of halo, —OH, C1-8alkyl, C2-8alkenyl, C1-8alkoxy, C2-8alkenyloxy, S(O)aR6, —NR7R8, —COR6, —COOR6, —R10COOR6, —OR10COOR6, —CONR7R8, —OC(O)R9, —R10NR7R8, —OR10NR7R8, nitro, and cyano, wherein a is 0, 1 or 2; R6 is selected from the group consisting of H, C1-8alkyl, C1-8alkoxy and C2-8alkenyl; each R7 and R8 is the same or different and is independently selected from the group consisting of H, C1-8alkyl, C2-8alkenyl and C3-8alkynyl; R9 is selected from the group consisting of H, C1-2alkyl and —NR7R8; and R10 is C1-8alkyl. As will be appreciated by those skilled in the art, the number of possible substituents on the cycloalkyl ring will depend upon the size of ring. In one embodiment, the cycloalkyl is a cyclohexyl which may be substituted as described above.

The term “aryl” refers to aromatic groups selected from the group consisting of phenyl, 1-naphthyl and 2-naphthyl. The term “aryl” also refers to substituted aryl wherein the phenyl or naphthyl ring bears one or more substituents selected from the group consisting of halo, —OH, C1-8alkyl, C2-8alkenyl, C1-8alkoxy, C2-8alkenyloxy, S(O)aR6, —NR7R8, —COR6, —COOR6, —R10COOR6, —OR10COOR6, —CONR7R8, —OC(O)R9, —R10NR7R8, —OR10NR7R8, nitro, and cyano, wherein a is 0, 1 or 2; R6 is selected from the group consisting of H, C1-8alkyl, C1-8alkoxy and C2-8alkenyl; each R7 and R8 is the same or different and is independently selected from the group consisting of H, C1-8alkyl, C2-8alkenyl and C3-8alkynyl; R9 is selected from the group consisting of H, C 1-8alkyl and —NR7R8; and R10 is C 1-8alkyl. As will be appreciated by those skilled in the art, the number of possible substituents on the aryl ring will depend upon the size of ring. For example, when the aryl ring is phenyl, the aryl ring may have up to 5 substituents selected from the foregoing list. One skilled in the art will readily be able to determine the maximum number of possible substituents for a 1-naphthyl or 2-naphthyl ring. One example of an aryl ring according to formula (II) is phenyl, which may be substituted as described above.

The term “heterocycle” refers to a monocyclic saturated or unsaturated non-aromatic carbocyclic rings and fused bicyclic non-aromatic carbocyclic rings, having the specified number of members in the ring and containing 1, 2 or 3 heteroatoms selected from N, O and S. Examples of particular heterocyclic groups include but are not limited to tetrahydrofuran, dihydropyran, tetrahydropyran, pyran, oxetane, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, tetrahydrothiophene, and the like. The term “heterocycle” also refers to substituted heterocycles wherein the heterocyclic ring bears one or more substituents selected from the group consisting of halo, —OH, C1-8alkyl, C2-8alkenyl, C1-8alkoxy, C2-8alkenyloxy, S(O)aR6, —NR7R8, —COR6, —COOR6, —R10COOR6, —OR10COOR6, —CONR7R8, —OC(O)R9, —R10NR7R8, —OR10NR7R8, nitro, and cyano, wherein a is 0, 1 or 2; R6 is selected from the group consisting of H, C1-8alkyl, C1-8alkoxy and C2-8alkenyl; each R7 and R8 is the same or different and is independently selected from the group consisting of H, C1-8alkyl, C2-8alkenyl and C3-8alkynyl; and R9 is selected from the group consisting of H, C1-8alkyl and —NR7R8; and R10 is C1-8alkyl. As will be appreciated by those skilled in the art, the number of possible substituents on the heterocyclic ring will depend upon the size of ring. There are no restrictions on the positions of the optional substituents in the heterocycles. Thus, the term encompasses rings having a substituent attached to the ring through a heteroatom. One skilled in the art will readily be able to determine the maximum number and locations of possible substituents for any given heterocycle. One example of a heterocycle according to the invention is piperidine, which may be substituted as described above.

The term “heteroaryl” refers to aromatic monocyclic heterocyclic rings and aromatic fused bicyclic rings having the specified number of members in the ring, having at least one aromatic ring and containing 1, 2 or 3 heteroatoms selected from N, O and S. Examples of particular heteroaryl groups include, but are not limited to, furan, thiophene, pyrrole, imldazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole. The term “heteroaryl” also refers to substituted heteroaryls wherein the heteroaryl ring bears one or more substituents selected from the group consisting of halo, —OH, C1-8alkyl, C2-8alkenyl, C1-8alkoxy, C2-8alkenyloxy, S(O)aR6, —NR7R8, —COR6, —COOR6, —R10COOR6, —OR10COOR6, —CONR7R8, —OC(O)R9, —R10NR7R8, —OR10NR7R8, nitro, and cyano, wherein a is 0, 1 or 2; R6 is selected from the group consisting of H, C1-8alkyl, C1-8alkoxy and C2-8alkenyl; each R7 and R8 is the same or different and is independently selected from the group consisting of H, C1-8alkyl, C2-8alkenyl and C3-8alkynyl; and R9 is selected from the group consisting of H, C1-8alkyl and —NR7R8; and R10 is C1-8alkyl. As will be appreciated by those skilled in the art, the number of possible substituents on the heteroaryl ring will depend upon the size of ring. There are no restrictions on the positions of the optional substituents in heteroaryls. Thus, the term encompasses rings having a substituent attached to the ring through a heteroatom. One skilled in the art will readily be able to determine the maximum number and locations of possible substituents for any given heteroaryl. A heteroaryl according to the invention is pyridine, which may be substituted as described above.

The term “protecting group” refers to suitable protecting groups useful for the synthesis of compounds of formula (I) wherein X is OH. Suitable protecting groups are known to those skilled in the art and are described in Protecting Groups in Organic Synthesis, 3rd Edition, Greene, T. W.; Wuts, P. G. M. Eds.; John Wiley & Sons: NY, 1999. Examples of protecting groups include but are not limited to methyl, ethyl, benzyl, substituted benzyl, and tert-butyl. In one embodiment the protecting group is methyl.

Example 16 of PCT/US01/27622 (Smith Kline Beecham plc) has the following structure of formula (IIa):

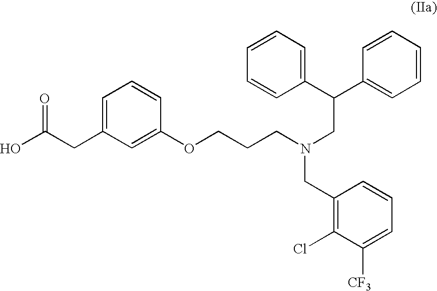

Compounds of formula (II) may be made according to any suitable method of organic chemistry. One method given in the specification is a solid phase synthesis process as depicted in Scheme 2. 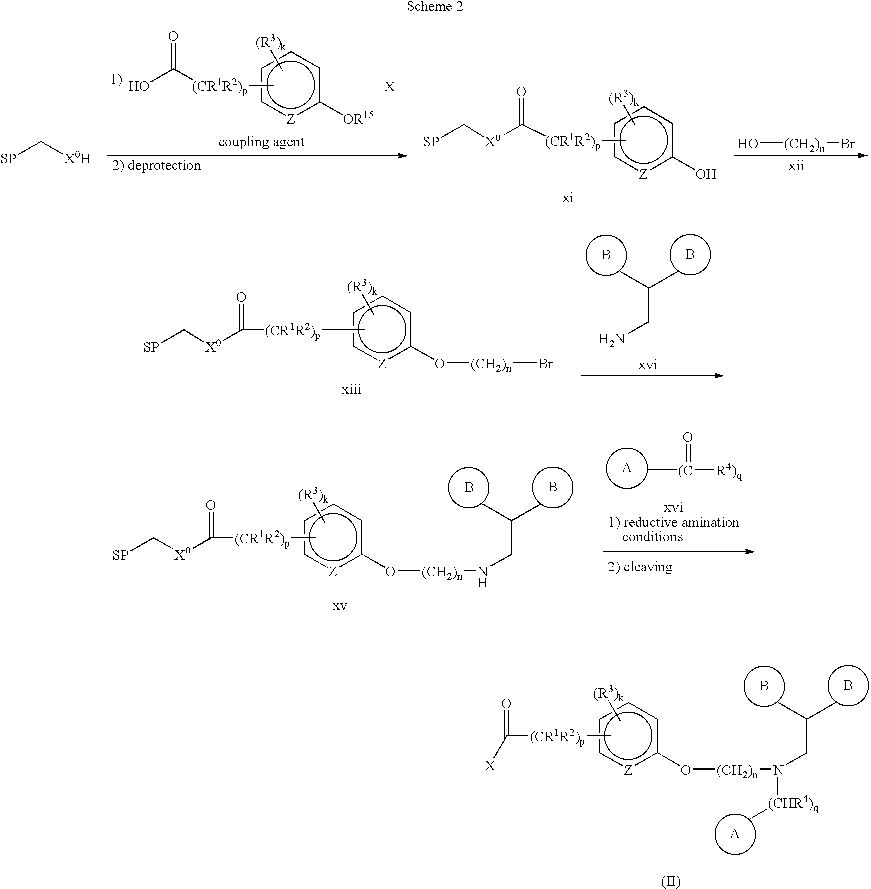
wherein X⁰ is —O— or —NH—, SP is solid phase, R¹⁵ is H or a protecting group, and all other variables are as defined above in connection with the description of compounds of formula (II).

In general, the reaction proceeds by a) reacting a solid phase-bound amine (where X in the compound of formula (II) is NH₂) or alcohol (where X in the compound of formula (II) is OH) with a compound of formula (x) and a coupling agent to produce a solid phase-bound compound of formula (xi); b) in the embodiment wherein R¹⁵ is a protecting group, deprotecting the solid phase bound compound to prepare the compound of formula (xi); c) alkylating the solid phase-bound compound of formula (xi) with an alcohol of formula (xii) to produce a solid phase-bound compound of formula (xiii); d) reacting the solid-phase-bound compound of formula (xiii) with a compound of formula (xiv) to produce the solid-phase bound compound of formula (xv); and e) reacting the solid phase-bound compound of formula (xv) with a compound of formula (xvi) under reductive amination conditions to produce the solid phase-bound compound of formula (II). The process may optionally further comprise the step of cleaving the solid phase-bound compound of formula (II) from the solid phase using conventional techniques such as treatment with mild acid.

Compounds of formula (II) are commercially available or may be prepared using conventional techniques such as those described in European Patent No. 303,742.

In one embodiment, LXR agonists of the present invention relates to a compound of formula (II), and the compound of formula (IIa).

Compounds of formula (II) are described in U.S. Provisional application Ser. Nos. 09/368,427, 60/368,425 and 60/368,426, each filed Mar. 27, 2002: 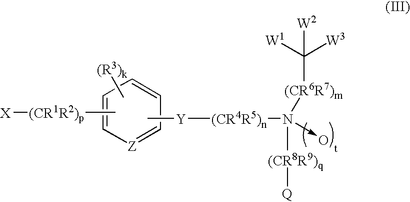
wherein:

X is selected from C₁-C₈ alkyl, halo, —OR¹⁰, —NR¹⁴R¹⁵, nitro, cyano, —COOR¹⁰, —COR¹³, —OCOR¹³, —CONR¹⁴R¹⁵, —N(R¹⁷)COR¹³, —N(R¹⁷)CONR¹⁴R¹⁵, —N(R¹⁷)COOR¹³, —SO₃H, —SO₂NR¹⁴R¹⁵, —C(═NR¹⁷)NR¹⁴R¹⁵, —N(R¹⁷)SO₂R¹⁶, and a 5 or 6-membered heterocyclic group;

or X and an adjacent R³, taken together with the atoms to which they are bonded, form an alkylenedioxy moiety;

Z is CH, CR³ or N, wherein when Z is CH or CR³, k is 0-4 and t is 0 or 1, and when Z is N, k is 0-3 and t is 0;

Y is selected from —O—, —S—, —N(R¹⁰)—, and —C(R⁴)(R⁵)—;

W¹ is selected from C₁-C₆ alkyl, C₃-C₈ cycloalkyl, aryl and Het, wherein said C₁-C₈ alkyl, C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;

p is 0-8;

n is 2-8;

m is 0 or 1;

q is 0 or 1;

t is 0 or 1;

each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where any of said C₁-C₆ alkyl is optionally unsubstituted or substituted by one or more halo substituents;

each R³ is the same or different and is independently selected from halo, cyano, nitro, C₁-C₆ alkyl, C₃-C₆ alkenyl, C₃-C₆ alkynyl, —C₀-C₆ alkyl-AR, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₁-C₆ alkyl is optionally unsubstituted or substituted by one or more halo substituents;

each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;

R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)ₓ—C₁-C₆ alkyl, —C₀-C₆ alkyl-S(O)ₓ—Ar, —C₀-C₆ alkyl-S(O)ₓ-Het, —C₀-C₆ alkyl-S(O)ₓ—C₃-C₇ cycloalkyl, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R¹⁶ is C₁-C₆ alkyl, —C₀-C₆ alkyl-Ar or —C₀-C₆ alkyl-Het; and

R¹⁷ is H, C₁-C₆ alkyl, —C₀-C₆ alkyl-Ar or —C₀-C₆ alkyl-Het.

Compounds of formula (IV) are described in U.S. Provisional Application No. 60/368,415, filed Mar. 27, 2002: 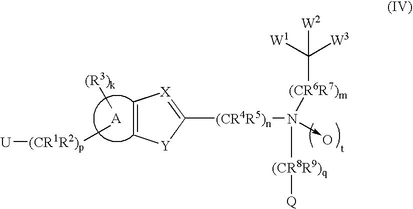
wherein:

X is CH or N;

Y is N(R¹⁰), O, or S, wherein t is 0 or 1 when Y is N(R¹⁰) or O, and t is 0 when Y is S;

U is selected from halo, —OR¹⁰, —NR¹⁴R¹⁵, nitro, cyano, —COOR¹⁰, —COR¹³, —OCOR¹³, —CONR¹⁴R¹⁵, —N(R¹⁴)COR¹³, —SO₃H, —SO₂NR¹⁴R¹⁵, —C(═NR¹⁷)NR¹⁴R¹⁵, —N(R¹⁴)SO₂R¹⁶, and a 5 or 6-membered heterocyclic group;

A is a phenyl fused ring moiety or a pyridyl fused ring moiety, wherein when A is a phenyl ring moiety, k is 0-3 and t is 0 or 1 and when A is a pyridyl ring moiety, k is 0-2 and t is 0;

W¹ is selected from C₃-C₈ cycloalkyl, aryl and Het, wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR13, where said C₁-C₆ alkyl, is optionally unsubstituted or substituted by one or more halo substituents;

W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;

p is 0-8;

n is 2-8;

m is 0 or 1;

q is 0 or 1;

t is 0 or 1;

each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where said C₁-C₆ alkyl is optionally unsubstituted or substituted by one or more halo substituents;

each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₁-C₆ alkyl is optionally unsubstituted or substituted by one or more halo substituents;

each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

each R^{1l} and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;

R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)ₓ—C₁-C₆ alkyl, —C₀-C₆ alkyl-S(O)ₓ—Ar, —C₀-C₆ alkyl-S(O)ₓ-Het, —C₀-C₆ alkyl-S(O)ₓ—C₃-C₇ cycloalkyl, —C₀-C₆ alkyl-NH—Ar, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R¹⁶ is C₁-C₆ alkyl, —C₀-C₆ alkyl-Ar or —C₀-C₆ alkyl-Het; and

R¹⁷ is H, C₁-C₆ alkyl, —C₀-C₆ alkyl-Ar or —C₀-C₆ alkyl-Het.

Unless otherwise provided, each alkyl, alkoxy, alkenyl, alkynyl, cycloalkyl, aryl or Het (including any 3-5-membered, 4-7-membered or 5-7-membered carbocyclic or heterocyclic rings or ring moieties) in the compounds of formula (III) and (IV) is independently unsubstituted or substituted with one ore more substituents defined hereinbelow.

In the compounds of formula (IV), group A is defined as a phenyl or a pyridyl fused ring moiety and is exemplified by the following:

Group A fused ring moiety:

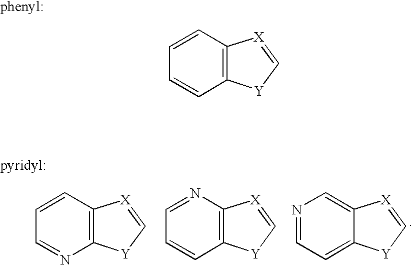

As used to define the compounds of formulas (III) or (IV), the term “alkyl” represents a straight-or branched-chain saturated hydrocarbon, containing 1 to 10 carbon atoms, unless otherwise provided, which may be unsubstituted or substituted by one or more of the substituents described below. Exemplary alkyls include, but are not limited to methyl (Me), ethyl (Et), n-propyl, isopropyl, n-butyl, isobutyl, t-butyl, n-pentyl, neopentyl and hexyl and structural isomers thereof. Any “alkyl” herein may be optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, and —CO₂H.

When combined with another substituent term as used to define the compounds of formulas (III) or (IV) (e.g., aryl or cycloalkyl as in -alkyl-Ar or -alkyl-cycloalkyl), the “alkyl” term therein refers to an alkylene moiety, that is, an unsubstituted divalent straight-or branched-chain saturated hydrocarbon moiety, containing 1 to 10 carbon atoms, unless otherwise provided. For example, the term “—C0-C6 alkyl-Ar”, where C is 1-6 is intended to mean the radical -alkyl-aryl (e.g., —CH2-aryl or —CH(CH3)-aryl) and is represented by the bonding arrangement present in a benzyl group. The term “C0 alkyl” in a moiety, such as —C0-C6 alkyl-Ar or —O—(C0-C6 alkyl)-Ar, provides for no alkyl/alkylene group being present in the moiety. Thus, when C is zero, —C0-C6 alkyl-Ar is equivalent to —Ar and —O—(C0-C6 alkyl)-Ar is equivalent to —O—Ar.

As used to define the compounds of formulas (III) or (IV), the term “alkenyl” represents a straight-or branched-chain hydrocarbon, containing 2 to 10 carbon atoms, unless otherwise provided, and one or more carbon-carbon double bonds; Alkenyl groups may be unsubstituted or substituted by one or more of the substituents described below. Exemplary alkenyls include, but are not limited ethenyl, 1-propenyl, 2-propenyl, 1-butenyl, 2-butenyl, isobutenyl, butadienyl, pentenyl and hexenyl and structural isomers thereof. Both cis (Z) and trans (E) isomers of each double bond that may be present in the compounds of formula (In) or (IV) are included within the scope of this definition. Any “alkenyl” herein may be optionally substituted by one or more of the substituents independently selected from the group halo, —OH, —SH, —NH2, —NH(unsubstituted C1-C6 alkyl), —N(unsubstituted C1-C6 alkyl)(unsubstituted C1-C6 alkyl), unsubstituted —OC1-C6 alkyl, and —CO2H.

As used to define the compounds of formulas (III) or (IV), the term “alkynyl” represents a straight- or branched-chain hydrocarbon, containing 2 to 10 carbon atoms, unless otherwise provided, and one or more carbon-carbon triple bonds and, optionally, one or more carbon-carbon double bonds. Both cis (Z) and trans (E) isomers of each double bond that may be present in the compounds of formula (III) or (IV) are included within the scope of this definition. Exemplary alkynyls include, but are not limited ethynyl, propynyl (propargyl, isopropynyl), 1-butynyl, 2-butynyl, 3-butynyl, pentynyl and hexynyl and structural isomers thereof. Any “alkynyl” herein may be optionally substituted by one or more of the substituents independently selected from the group halo, —OH, —SH, —NH2, —NH(unsubstituted C1-C6 alkyl), —N(unsubstituted C1-C6 alkyl)(unsubstituted C1-C6 alkyl), unsubstituted —OC1-C6 alkyl, and-CO2H.

As used to define the compounds of formulas (III) or (IV), when an alkenyl or alkynyl group is a substituent on an oxygen, nitrogen or sulfur atom (e.g., as in oxy (—OR), thio (—SR), ester (—CO2R or —C(O)SR), amino (—NRR) or amido (—CONRR) moieties and the like), it is understood that a double or triple bond of the alkenyl or alkynyl group is not located on carbons that are α,βto the oxygen, nitrogen or sulfur atom. Compounds containing ene-amino or enol-type moieties (—NR—CR═CR— or —O—CR═CR—) are not intended to be included within the scope of the definition of the compounds of formula (III) or (IV).

As used to define the compounds of formulas (III) or (IV), the term “cycloalkyl” represents a non-aromatic monocyclic, bicyclic, or tricyclic hydrocarbon containing from 3 to 10 carbon atoms which may be unsubstituted or substituted by one or more of the substituents described below and may be saturated or partially unsaturated. Exemplary cycloalkyls include monocyclic rings having from 3-7 or 3-6, carbon atoms, such as cyclopropyl, cyclobutyl, cyclopentyl, cyclopentenyl, cyclopentadienyl, cyclohexyl, cyclohexenyl and cycloheptyl. Any “cycloalkyl” herein may be optionally substituted by one or more of the substituents independently selected from the group halo, cyano, C1-C6 alkyl (which specifically includes C1-C6 haloalkyl, —C0-C6 alkyl-OH, —C0-C6 alkyl-SH and —C0-C6 alkyl-NR′R″), C3-C6 alkenyl, oxo, —OC1-C6alkyl, —OC1-C6 alkenyl, —C0-C6 alkyl-COR′, —C0-C6 alkyl-CO2R′, —C0-C6 alkyl-CONR′R″, —OC0—C6 alkyl-CO2H, —OC2—C6 alkyl-NR′R″, and —C0-C6 alkyl-SO2NR′R″, wherein each R′ and R″ are independently selected from H or unsubstituted C1-C6 alkyl.

As used to define the compounds of formulas (III) or (IV), the terms “Ar” or “aryl” is used interchangeably at all occurrences mean a substituted or unsubstituted carbocyclic aromatic group, which may be optionally fused to another carbocyclic aromatic group moiety or to a cycloalkyl group moiety, which may be optionally substituted or unsubstituted. Examples of suitable Ar or aryl groups include phenyl, naphthyl indenyl, 1-oxo-1H-indenyl and tetrahydronaphthyl. Any “Ar”, “aryl” or “phenyl” herein may be optionally unsubstituted or substituted by one or more of the substituents independently selected from the group halo, cyano, C1-C6 alkyl (which specifically includes C1-C6 haloalkyl, —C0-C6 alkyl-OH, —C0-C6 alkyl-SH and —C0-C6 alkyl-NR′R″), C3-C6 alkenyl, —OC1-C6alkyl, —OC1-C6 alkenyl, —C0-C6 alkyl-COR′, —C0-C6 alkyl-CO2R′, —C0-C6 alkyl-CONR′R″, —OC0—C6 alkyl-CO2H, —OC2—C6 alkyl-NR′R″, —C0-C6 alkyl-C(═NR′)NR′R″, and —C0-C6 alkyl-SO2NR′R″, wherein each R′ and R″ are independently selected from H or unsubstituted C1-C6 alkyl.

As used to define the compounds of formulas (III) or (IV), the term “Het” means a stable 5- to 7-membered monocyclic, a stable 7- to 10-membered bicyclic, or a stable 11- to 18-membered tricyclic heterocyclic ring group, all of which are saturated, unsaturated or aromatic, and consist of carbon atoms and from one to three heteroatoms selected from the group consisting of N, O and S, and which includes bicyclic and tricyclic rings containing one or more fused cycloalkyl, aryl (e.g., phenyl) or heteroaryl (aromatic Het) ring moieties. As used herein the term “Het” is also intended to encompass heterocyclic groups containing nitrogen and/or sulfur where the nitrogen or sulfur heteroatoms are optionally oxidized or the nitrogen heteroatom is optionally quaternized. The heterocyclic group may be attached at any heteroatom or carbon atom that results in the creation of a stable structure. Any “Het” herein may be optionally unsubstituted or substituted by one or more of the substituents independently selected from the group halo, cyano, C1-C6 alkyl (which specifically includes C1-C6 haloalkyl, —C0-C6 alkyl-OH, —C0-C6 alkyl-SH and —C0-C6 alkyl-NR′R″), C3-C6 alkenyl, oxo, —OC1-C6alkyl, —OC1-C6 alkenyl, —C0-C6 alkyl-COR′, —C0-C6 alkyl-CO2R′, —C0-C6 alkyl-CONR′R″, —OC0—C6 alkyl-CO2H, —OC2-C6 alkyl-NR′R″, —C0-C6 alkyl-C(═NR′)NR′R″ and —C0-C6 alkyl-SO2NR′R″, wherein each R′ and R″ are independently selected from H or unsubstituted C1-C6 alkyl.

Examples of such heterocyclic groups include, but are not limited to piperidinyl, piperazinyl, 2-oxopiperazinyl, 2-oxopiperidinyl, 2-oxopyrrolodinyl, 2-oxoazepinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, 1,3-benzodioxolyl (e.g., methylenedioxy-substituted phenyl), 1,4-benzodioxolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothienyl, dihydrobenzofuranyl, dihydrobenzothienyl, dihydroindolyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable.

Examples of the 4-7 membered heterocyclic rings useful in the compounds of formula (III) or (IV), include, but are not limited to azetidinyl, piperidinyl, piperazinyl, 2-oxopiperazinyl, 2-oxopiperidinyl, 2-oxopyrrolodinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 4-7 membered heterocyclic group may be optionally unsubstituted or substituted by one or more of the substituents independently selected from the group halo, cyano, C1-C6 alkyl (which specifically includes C1-C6 haloalkyl, —C0-C6 alkyl-OH, —C0-C6 alkyl-SH and —C0-C6 alkyl-NR′R″), C3-C6 alkenyl, oxo, —OC1-C6alkyl, —OC1-C6 alkenyl, —C0-C6 alkyl-COR′, —C0-C6 alkyl-CO2R′, —C0-C6 alkyl-CONR′R″, —OC0—C6 alkyl-CO2H, —OC2—C6 alkyl-NR′R″, —C0-C6 alkyl-C(═NR′)NR′R″ and —C0-C6 allyl-SO2NR′R″, wherein each R′ and R″ are independently selected from H or unsubstituted C1-C6 alkyl.

Examples of 5 or 6 membered heterocyclic groups include, but are not limited to piperidinyl, piperazinyl, 2-oxopiperazinyl, 2-oxopiperidinyl, 2-oxopyrrolodinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 5-6 membered heterocyclic group may be attached at any heteroatom or carbon atom that results in the creation of a stable structure. The 5-6 membered heterocyclic group may be optionally unsubstituted or substituted by one or more of the substituents independently selected from the group halo, cyano, C1-C6 alkyl (which specifically includes C1-C6 haloalkyl, —C0-C6 alkyl-OH, —C0-C6 alkyl-SH and —C0-C6 alkyl-NR′R″), C3-C6 alkenyl, oxo, —OC1-C6alkyl, —OC1-C6 alkenyl, —C0-C6 alkyl-COR′, —C0-C6 alkyl-CO2R′, —C0-C6 alkyl-CONR′R″, —OC0—C6 alkyl-CO2H, —OC2—C6 alkyl-NR′R″, —C0-C6 alkyl-C(═NR′)NR′R″ and —C0-C6 alkyl-SO2NR′R″, wherein each R′ and R″ are independently selected from H or unsubstituted C1-C6 alkyl.

In the compounds of formulas (III) and (IV), the terms “halogen” and “halo” represent chloro, fluoro, bromo or iodo substituents; “alkoxy” is intended to mean the radical —ORa, where Ra is an alkyl group, wherein alkyl is as defined above, provided that —O—C1 alkyl may be optionally substituted by one or more of the substituents independently selected from the group halo and —CO2H. (exemplary alkoxy groups include methoxy, ethoxy, propoxy, and the like); “phenoxy” is intended to mean the radical —ORar, where Rar is a phenyl group; “acetoxy” is intended to mean the radical —O—C(═O)-methyl; “benzoyloxy” is intended to mean the radical —O—C(═O)-phenyl; and “oxo” is intended to mean the keto diradical ═O, such as present on a pyrrolidin-2-one ring.

A method for the preparation of compounds of formula (III), comprises the steps of: (a)

reacting an alcohol having the formula: HY′-(CR4R5)n-L, where Y′ is —O—, —S—, —NH or protected —NH and L is a leaving group, such as a halogen (iodide, bromide or chloride), sulfonate (tosylate, mesylate, triflate, etc.) or is a group that is converted to a leaving group (e.g., an alcohol), with an alcohol having the formula: 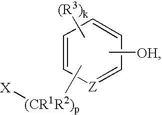
where X is a protected carboxylic acid moiety, to form a compound having the formula:

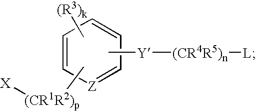

(b) reacting the compound formed in step (a) with a secondary amine having the formula 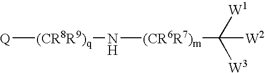
to form a compound having the formula:

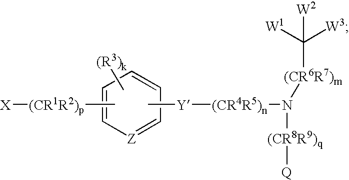

(c) converting the protected carboxylic acid moiety into a desired amide moiety; and

(d) optionally oxidizing the compound. formed in step (b) to the N-oxide thereof.

Another method for the preparation of compounds of formula (III), comprises the steps of:

(a) reacting an acetylene having the formula: R′O—(CR⁴R⁵)ₙ₋₁—C═C—H, where R′ is a hydroxyl protecting group, with a halogen-containing aromatic compound having the formula 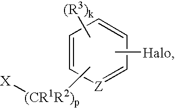
where X is a protected carboxylic acid moiety and Halo is bromo or iodo, in the presence of a catalyst to form a compound having the formula:

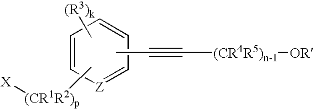

(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol), to form a compound having the formula:

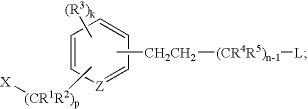

(c) reacting the compound formed in step (b) with an amine having the formula: 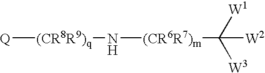
to form a compound having the formula:

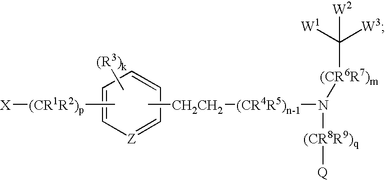

(d) converting the protected carboxylic acid moiety into a desired amide moiety; and

(e) optionally oxidizing the compound. formed in step (b) to the N-oxide thereof.

Another method for the preparation of compounds of formula (M), comprises the steps of:

(a) reacting an alcohol having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), with a compound having the formula: 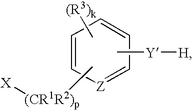
where Y′ is —O—, —S—, or —NH— and X is defined as above or a protected form thereof, to form a compound having the formula:

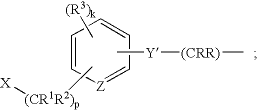

(b) reacting the compound formed in step (a) with a secondary amine having the formula 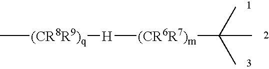
to form a compound having the formula:

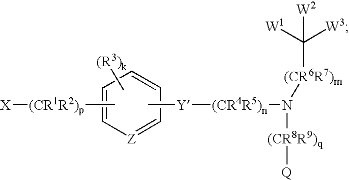

(c) removing any protecting groups; and

(d) optionally oxidizing the compound formed in step (b) or (c)to the N-oxide thereof.

Another method for the preparation of compounds of formula (III), comprises the steps of:

(a) reacting a compound having the formula: 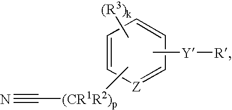
where Y′ is —O—, —S—, or —NH— and R′ is a suitable protecting group for —OH, —SH, or —NH₂, with a hydrazide or azide to form a heterocyclic-containing compound having the formula: 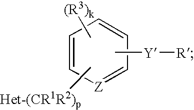
(b) optionally protecting the NH moiety of the heterocyclic group with a protecting group, and removing the R′ protecting group;

(c) reacting the compound formed in step (b) with a compound having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), to form a compound having the formula: 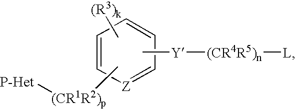
where P is an optional protecting group or H;

(d) reacting the compound formed in step (c) with an amine having the formula: 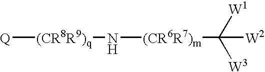
to form a compound having the structure:

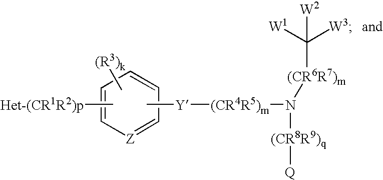

(e) removing any protecting groups.

Another method for the preparation of compounds of formula (III), comprises the steps of:

(a) reacting an acetylene having the formula: R′O—(CR⁴R⁵)ₙ₋₁—C═C—H, where R′ is a hydroxyl protecting group, with a halogen-containing aromatic compound having the formula 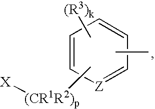
where Halo is bromo or iodo, in the presence of a catalyst to form a compound having the formula: 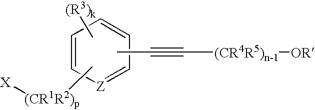
(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol) to form a compound having the formula:

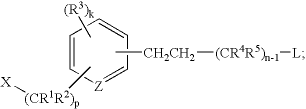

(c) reacting the compound formed in step (b) with an amine having the formula: 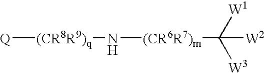
to form a compound having the formula:

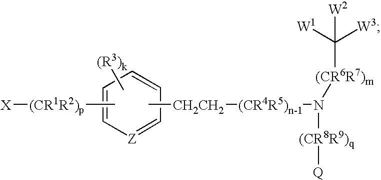

(d) removing any protecting groups; and

(e) optionally oxidizing the compound formed in step (c) or (d) to the N-oxide thereof.

Another method for the preparation of compounds of formula (III), comprises the steps of:

(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol) with a phenol having the formula: 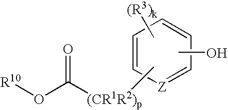
to form an aryl ether having the formula:

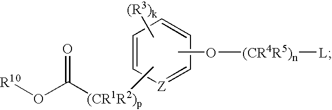

(b) reacting an amine having the formula 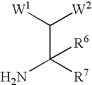
with and an aldehyde having the formula Q-CHO or a ketone to form a secondary amine having the formula:

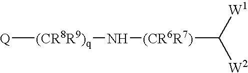

(c) reacting the ether formed in step (a) with the secondary amine formed in step (b) to form a compound of this invention having the formula:

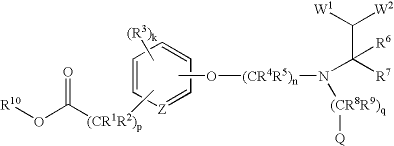

(d) when R¹⁰ is other than H, optionally converting the compound. formed in step (c) to the compound of this invention, wherein R¹⁰ is H.

Another method for the preparation of compounds of formula (III), comprises the steps of:

(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol), with an amine having the formula: 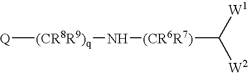
to form a tertiary amine having the formula: 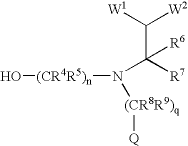
(b) reacting the tertiary amine formed in step (a) with a phenol having the formula: 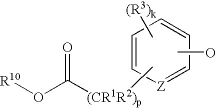
to form a compound of this invention having the formula: 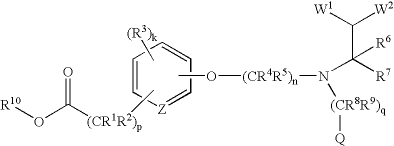
(c) when R¹⁰ is other than H, optionally converting the compound. formed in step (b) to the compound of this invention, wherein R¹⁰ is H.

Another method for the preparation of compounds of formula (III), comprises the steps of:

(a) reacting an alcohol having the formula: HO—(CR⁴R⁵)ₙ-L, where L is a leaving group, such as a halogen (iodide, bromide or chloride) or sulfonate (tosylate, mesylate, triflate, etc.), with a phenol having the formula: 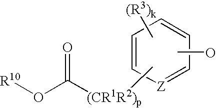
to form an ether-alcohol having the formula: 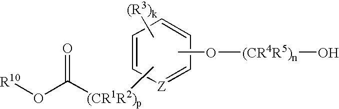
(b) converting alcohol moiety of the ether-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol) and treating the resulting compound with an amine having the formula: 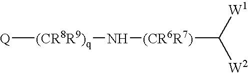
to form a compound of this invention having the formula:

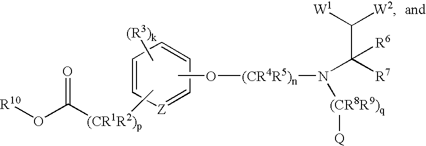

(c) when R¹⁰ is other than H, optionally converting the compound. formed in step (b) to the compound of this invention, wherein R¹⁰ is H.

The method for the preparation of compounds of formula (IV), comprises the steps of:

(a) coupling an acetylene having the formula: with a phenol having the formula: 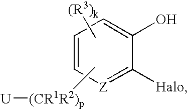
where Halo is a halogen selected from iodo or bromo, in the presence of a metal catalyst to form an aryl-alcohol having the formula:

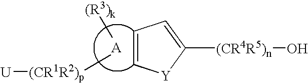

(b) converting alcohol moiety of the aryl-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol), and treating the resulting compound with an amine having the formula: 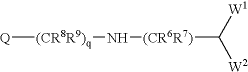
to form the compound of formula (IV);

(c) optionally converting the compound of formula (IV)from step (b) into another compound of formula (IV); and

(d) optionally oxidizing the compound. formed in step (c) to the N-oxide thereof.

Alternatively, the compounds of formula (IV) may be prepared by

(a) coupling an acetylene having the formula: with a phenol having the formula: 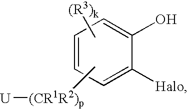
where Halo is a halogen selected from iodo or bromo, in the presence of a metal catalyst to form an aryl-alcohol having the formula:

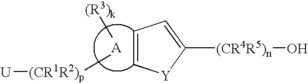

(b) converting alcohol moiety of the aryl-alcohol formed in step (a) into L′, where L′ is a leaving group such as a halogen (iodide, bromide or chloride) or a sulfonate(tosylate, mesylate, triflate, etc.) and treating the resulting compound with sodium azide, followed by hydrogenation in the presence of a palladium catalyst to form a primary amine having the formula:

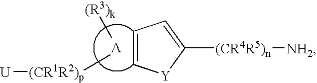

(c) treating the primary amine with a first aldehyde in the presence of a reducing agent, to form a secondary amine and treating the secondary amine with a second aldehyde in the presence of a reducing agent to form the compound of formula (IV);

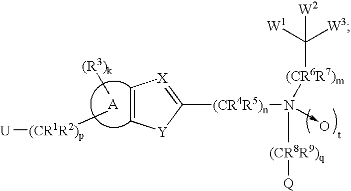

(d) optionally converting the compound of formula (IV) from step (b) into another compound of formula (IV); and

(e) optionally oxidizing the compound. formed in step (b) or (c) to the N-oxide thereof.

International Patent Applications WO 01/41704 (Merck & Co., Inc.) discloses compound of formula (V) 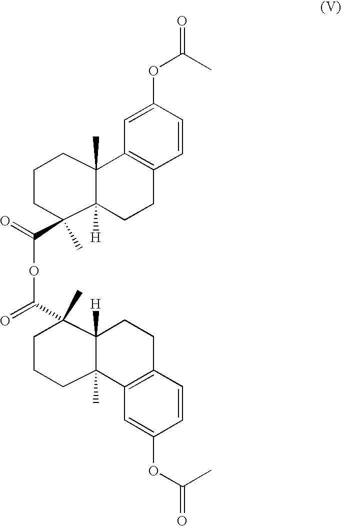
as being an agonist of LXR and its use in pharmaceutical formulations to prevent and treat atherosclerotic diseases and related diseases.

Other LXR agonists may be identified by assays such as those described in the above referenced patent applications, for example, the assays described in Examples 1 and 2 of PCT/US01/27622. Biotinylated LXRβ protein was incubated for 20-25 minutes at a concentration of 25 nM in assay buffer (50 mM KCl, 50 mM Tris-pH8, 0.1 mg/ml FAF-BSA, 10 mM DTT) with equimolar amounts of streptavidin-AlloPhycoCyanin (APC, Molecular Probes). At the same time, the biotinylated peptide comprising amino acids 675-699 of SRC-1 (CPSSHSSLTERHKILHRLLQEGSPS-CONH2) (SEQ ID NO: 5) at a concentration of 25 nM was incubated in assay buffer with a ½ molar amount of streptavidin-labelled Europium (Wallac) for 20-25 minutes. After the initial incubations are completed, a 10 molar excess (250 nM) of cold biotin was added to each of the solutions to block the unattached streptavidin reagents. After 20 min at room temp, the solutions were mixed yielding a concentration of 12.5 nM for the dye-labelled LXRβ protein and SRC-1 peptide. 80 μL of the protein/peptide mixture was added to each well of an assay plate containing 20 μL of test compound. The final volume in each well was 0.1 mL, and the concentration in the well for the dye-labelled protein and peptide was 10 nM. The final test compound concentrations were between 56 pM and 10 μM. The plates were incubated at room temp in the dark for 4-12 hours and then counted on a Wallac Victor fluorescent plate reader. In this assay 1 μM 24(S), 25-epoxycholesterol gave a reading of 20000 fluorescence units over a background reading of 10000 fluorescence units. The assay for LXRα was run according to the procedures described above using his-tagged LXRα ligand binding domain (amino acids 183-447 of Genbank accession number U22662, with the 14^{th} amino acid corrected to A from R).

Suitable pharmaceutically acceptable salts include salts of salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glutarate and a-glycerophosphate.

The LXR agonists referred to herein are conveniently prepared according to the methods disclosed in the above mentioned patent publications in which they are disclosed.

The salts and/or solvates of the LXR agonists may be prepared and isolated according to conventional procedures for example those disclosed in the, above mentioned, patent publications.

In the above mentioned method the LXR agonist, may be administered per se or as a pharmaceutical composition/formulation also comprising a pharmaceutically acceptable carrier.

In the treatment of the invention, the LXR agonist mentioned herein is formulated and administered in accordance with the methods disclosed in the above mentioned patent applications and patents.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Suitable compositions for oral administration may be unit dosage forms such as tablets and capsules. Other fixed unit dosage forms, such as powders presented in sachets, may also be used.

In accordance with conventional pharmaceutical practice, the carrier may comprise a diluent, filler, disintegrant, wetting agent, lubricant, colourant, flavourant or other conventional adjuvant.

Carriers may include, for example, microcrystalline cellulose, starch, sodium starch glycollate, polyvinylpyrrolidone, polyvinylpolypyrrolidone, magnesium stearate, sodium lauryl sulphate or sucrose.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, may be either suspended or dissolved in the vehicle. In preparing solutions the compound may be dissolved in water for injection and filter sterilized before filling into a suitable vial or ampoule and sealing. Advantageously, adjuvants such as a local anaesthetic, a preservative and buffering agents may be dissolved in the vehicle. To enhance the stability, the composition may be frozen after filling into the vial and the water removed under vacuum. Parenteral suspensions are prepared in substantially the same manner, except that the compound is suspended in the vehicle instead of being dissolved, and sterilization cannot be accomplished by filtration. The compound may be sterilized by exposure to ethylene oxide before suspending in the sterile vehicle. A surfactant or wetting agent may be included in the composition to facilitate uniform distribution of the compound.

Compositions may contain from 0.1% to 99% by weight or from 10-60% by weight, of the active material, depending upon the method of administration.

The compositions are formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

A therapeutically effective amount of LXR agonist of the present invention for preventing or treating cardiovascular pathology may depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

An LXR agonist agent may be given in the range of 0.1 to 100 mg/kg body weight of recipient (mammal) per day or in the range of 1 to 30 mg/kg body weight per day. Acceptable daily dosages of the LXR agonist for preventing/treating cardiovascular pathology may be from about 0.1 to about 1000 mg/day, or from about 0.2 to about 100 mg/day.

Thus, in one embodiment of the present invention a method is provided for treating or preventing cardiovascular pathology; comprising, administering a therapeutically effective amount of LXR agonist, or a pharmaceutically acceptable salt, solvate, or physiologically functional derivative thereof. In another aspect, the cardiovascular pathology is selected from the group consisting of cardiac hypertrophy, coronary heart disease, arrhythmia, restricted coronary blood flow, arteriosclerosis, heart failure, congestive heart failure (CHF), and myocardial infarction.

In another embodiment of the present invention a pharmaceutical composition is provided for treating or preventing cardiovascular pathology comprising a therapeutically effective amount of LXR agonist, or a pharmaceutically acceptable salt, solvate, or physiologically functional derivative thereof, and a pharmaceutically acceptable carrier. In another aspect the cardiovascular pathology is selected from the group consisting of cardiac hypertrophy, coronary heart disease, arrhythmia, restricted coronary blood flow, arteriosclerosis, heart failure, congestive heart failure (CHF), and myocardial infarction.

The following Examples are intended for illustration only and are not intended to limit the scope of the invention in any way; the present invention being defined by the appended claims.

### EXAMPLES

### Example 1

### 2-(3-{3-[[2-Chloro-3-(Trifluoromethyl)Benzyl](2,2-Diphenylethyl)Amino]Propoxy}-Phenyl)Acetic Acid (Formula IIa)

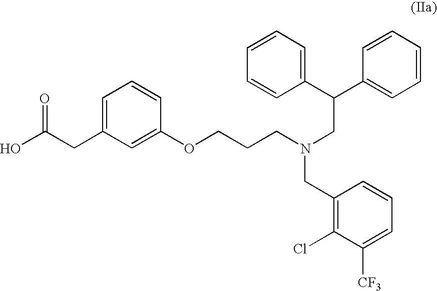

Argogel-MB-OH (6.0 g, 2.40 mmol, Argonaut Technologies) was treated with a solution of (3-{[tert-butyl(dimethyl)silyl]oxy}phenyl)acetic acid (5.40 g, 19.2 mmol, Eur. Pat. Appl. (1987) Application: EP 87-303742 19870428) in 50 mL of anhydrous dichloromethane followed by dicyclohexylcarbodiimide (4.16 g, 19.2 mmol) and 4-dimethylaminopyridine (2.50 g, 19.2 mmol). After rotating at room temperature for 15 hours, the resin was filtered, washed sequentially with dichloromethane (2×25 mL), dimethylformamide (2×25 mL), dichloromethane (3×25 mL), methanol (3×25 mL), dichloromethane (3×25 mL) and diethyl ether (2×25 mL). After drying under house vacuum overnight at 40° C., the resin was treated with 1.0 M tetrabutylammonium fluoride (24 mL, 23.4 mmol) in tetrahydrofuran, and the mixture was rotated for 4 hours. The resin was filtered, washed sequentially with dichloromethane (2×25 mL), dimethylformamide (2×25 mL), dichloromethane (3×25 mL), methanol (3×25 mL), and dichloromethane (3×25 mL) to give the deprotected phenol. The dry resin was treated with 90 mL of anhydrous toluene followed by triphenylphosphine (15.8 g, 60.0 mmol) and 3-bromo-1-propanol (8.4 g, 60.0 mmol). Upon cooling to 0° C., diisopropyl azodicarboxylate (12.1 g, 60.0 mmol) in 20 mL of anhydrous toluene was added in a dropwise fashion. The reaction was allowed to warm to room temperature and stirred for 15 hours. The resin was filtered, washed sequentially with dichloromethane (2×50 mL), dimethylformamide (2×50 mL), dichloromethane (3×50 mL), methanol (2×50 mL) and dichloromethane (3×50 mL), and dried under house vacuum. The bromide functionalized resin was treated with a solution of diphenethylamine (25.0 g, 127 mmol) in 60 mL of anhydrous dimethylsulfoxide, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50 mL), and dried under house vacuum at 40° C. The secondary amine resin (5.75 g, 2.0 mmol) was treated with a solution of 2-chloro-3-trifluoromethylbenzaldehyde (8.32 g, 40.0 mmol) in 80 mL of 8% acetic acid in dimethylformamide. Solid sodium triacetoxyborohydride (8.5 g, 40.0 mmol) was added, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50mL), and dried under house vacuum overnight at 50° C. The resin-bound product was treated with 30 mL of trifluoroacetic acid/dichloromethane (15/85) for 15 minutes, and the filtrate was collected. The cleavage procedure was repeated again, and the combined filtrates were concentrated under reduced pressure. The crude product was purified by preparative thin layer chromatography (silica gel, 1 mm plates, Merck 20×20 cm silica gel 60 F₂₅₄) eluting with methanol:dichloromethane (3:97) to give 7.0 mg of the title compound (5% yield based on theoretical loading of secondary amine resin) of a viscous oil: ¹H NMR (CDCl₃, 400 MHz) δ 7.42 (d, 1H, J=7.6), 7.23-7.10 (m, 12H), 6.85 (t, 2H, J=8.1), 6.63 (s, 1H), 6.61 (s, 1H), 4.11 (t, 1H, J=7.8), 3.75 (s, 2H), 3.63 (t, 2H, J=6.0), 3.59 (s, 2H), 2.12 (d, 2H, J=7.8), 2.67 (t, 2H, J−6.6), 1.81 (tt 2H, J=6.2); MS (ESP+) m/e 582 (MH⁺); TLC (EtOAc:hexanes/1:1) R_{f}=0.58.

### Example 2

### N-(2,2,2-Trifluoroethyl)-N-[4-(2,2,2-Trifluoro-1-Hydroxy-1-Trifluoromethyl-Ethyl)-Phenyl]-Benzenesulfonamide (Formula Ia)

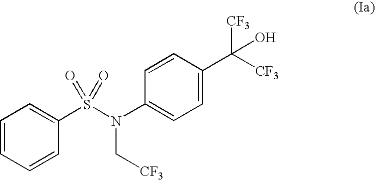 Preparation of N-trifluoroethylaniline derivative. 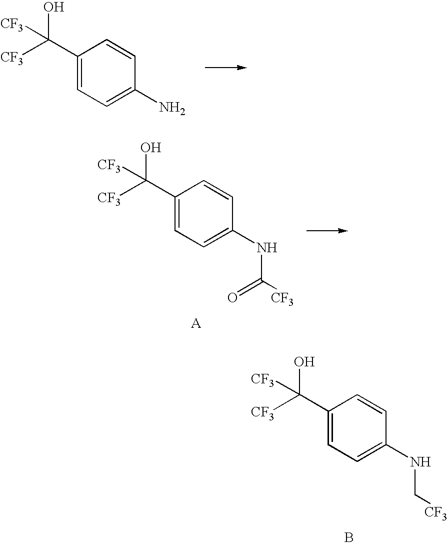
To a suspension of 4-[2,2,2-triflouro-1-hydroxy-1-(trifluoromethyl)ethyl]aniline (9.07 g, 35.0 mmol) in CH₂CL₂ (100 ml) was added to solution of trifluoroacetic anhydride (5.7 ml, 40.2 mmol) in CH₂Cl₂ (50 ml) dropwise at room temperature. The solution was stirred for 3 hours, the solution cleared and TLC indicated that the reaction was completed. The reaction mixture was washed with water, aqueous NaHCO₃, and brine. The organic layer was drawn off, dried over MgSO₄, filtered and concentrated to give 12.1 g of the intermediate trifluoroacetanitrilide (A). The intermediate A was taken up in the THF (50 ml) and treated with LiAlH₄ (4.00 g, 106 mmol) at refklux for 10 hours. The reaction was quenched sequentially adding 4 ml of water, 4 ml of 15% NaOH and 12 ml of water. The resulting suspension was stirred for an additional 30 minutes, filtered through a celite pad, which was then rinsed with THF. The combined filtrate and rinse was concentrated under reduced pressure. The residue was taken up in EtOAc, washed with Brine, dried over MgSO₄, filtered and concentrated. The resulting crude product was purified by chromatography on SiO₂ (4:1 hexane:EtOAc as eluant) to provide 11.og (92%) of the title compound (B).

¹H NMR (CDCl₃): δ 7.52 (J=8.6 Hz, 2H), 6.72 (d, J=8.6 Hz, 2H), 4.10 (bs, 1H), 3.80 (q, J=8.5 Hz, 2H), 3.31 (bs, 1H). MS (ES+): 342 (M+H, 100).

Sulfonylation of B

A sample of B from above (1.87 g, 5.48 mmol) was treated with benzenesulfonylchloride (1.18 g, 6.68 mmol) in pyridine (10 ml) at room temperature for 10 days. The reaction mixture as diluted with EtOAc, washed with aqueous NaHCO₃, and brine. The organic layer was dried over MgSO₄, filtered and concentrated. The crude product was purified by chromatography on SIO₂(4:1 hexane:EtOAc as eluant) to provide 1.65 g (62%) of compound Formula Ia.

¹H NMR (CDCl₃): δ 7;78 (J=8.8 Hz, 2H), 7.61 (t, J=7.6 Hz, 1H), 7.58 (d, J=7.6 Hz, 2H), 7.46 (t, J=Hz, 2H), 4.24 (q, J=8.2 Hz, 2H), 3.41 (s, 1H). MS (ES-): 480 (M-H, 100). Anal. Calcd. for C₁₇H₁₂F₉NO₃S:C, 42.42; H, 2.51; N, 2.91; S, 6.66. Found: C, 42.70; H, 2.55; N, 2.84; S, 6.61.

### Example 3

### Effect of Formula IIa on LV Mass

Male CD-1 mice (n=7 per group), 20-25 g in weight, were used for the experiment. Each mouse was anaesthetized with isoflurane 1-2%. Each mouse's aorta was exposed between the two renal arteries and was surgically constricted using a 30-gauge needle and 6.0 suture to induce heart growth. Seven days after surgery, baseline echocardiography was performed on each mouse to establish left ventricular mass (“LV mass”).

Enalapril, the gold standard in treating cardiac hypertrophy, was orally administered to the mice at the dose of 10 mg/kg beginning on day 11 (4 days after baseline) and was continued once daily for 10 days. The compound of Formula IIa was suspended in 0.5% methylcellulose (MC) solution (vehicle) and was orally administered to the mice at the doses of 10 mg/kg twice a day, beginning on day 11. The control group was administered with vehicle (0.5% MC) beginning on day 11. Repeat echocardiography was conducted on each mouse 14 and 21 days after surgery.

A statistically significant reduction (p<0.05) in LV Mass compared to vehicle was observed in the Formula IIa treatment group 14 and 21 days after surgery. Furthermore, Formula IIa (10 mg/kg b.i.d.) treatment prevented any further increase in LV Mass.

Enalapril (10 mg/kg) significantly (p<0.05) reduced LV Mass compared to vehicle at days 14 and 21 and also significantly (p<0.05) reversed the pathology (i.e., reduced LV Mass to less than baseline hypertrophy).

Table 1 shows mear±SE LV mass of mice treated with vehicle, Formula IIa, and enalapril at baseline (7 days after surgery), 14 and 21 days after surgery.

TABLE 1
Mean ± SE LV Mass of mice treated with vehicle,
Formula IIa, or enalapril

| | Mean ± SE LV Mass | | |
|---|---|---|---|
| Days after | | Formula IIa | Enalapril |
| Surgery | Vehicle (control) | 10 mg/kg BID | 10 mg/kg |
| 7 (Baseline) | 105.03 ± 3.08 | 100.88 ± 2.77 | 94.09 ± 1.950 |
| 14 | 121.07 ± 3.21 | 99.45 ± 1.98* | 84.90 ± 1.11*# |
| 21 | 134.12 ± 3.37 | 100.66 ± 2.43* | 83.07 ± l.13*# |

*p < 0.05 compared with vehicle at the same time point
#p < 0.05 reduced pathology compared with baseline

### Example 4

### Effect of Formula IIa, Formula Ia on LV Mass

Male CD-1 mice (n=7 per group), 20-25 g in weight, were used for the experiment. Each mouse was anaesthetized with isoflurane 1-2%. Each mouse's aorta was exposed between the two renal arteries and was surgically constricted using a 30-gauge needle and 6.0 suture to induce heart growth. Seven days after surgery, baseline echocardiography was performed on each mouse to establish left ventricular mass (“LV mass”).

Enalapril was orally administered to the mice at the dose of 10 mg/kg beginning on day 11 (4 days after baseline) and was continued once daily for 10 days. Formula IIa, suspended in 0.5% MC (vehicle), was orally administered to the mice at the doses of 3.0 mg/kg, 10 mg/kg, or 30 mg/kg once a day beginning on day 11. Formula Ia, suspended in 0.5% MC (vehicle), was orally administered to the mice at the dose of 50 mg/kg once a day beginning on day 11. The control group was administered with vehicle (0.5% MC). Repeat echocardiography was conducted on each mouse 14 and 21 days after surgery.

A statistically significant reduction (p<0.05) in LV Mass compared to vehicle was observed in all treatment groups 14 and 21 days after surgery compared with vehicle as shown in Table 2.

Table 2 shows mean±SE LV mass of mice treated with vehicle, Formula IIa, Formula Ia or enalapril at baseline (7 days after surgery), 14 and 21 days after surgery.

TABLE 2
Mean ± SE LV Mass of mice treated with vehicle, Formula IIa,
Formula Ia or enalapril

| Days | | Mean ± SE LV Mass | | | | |
|---|---|---|---|---|---|---|
| after | Vehicle | Formula IIa | | | | Enalapril |
| Surgery | (control) | 3 mg/kg | 10 mg/kg | 30 mg/kg | Formula Ia | 10 mg/kg |
| 7ⁿ | 97.69 ± 1.97 | 98.9 ± 4.59 | 98.01 ± 2.39 | 102.42 ± 2.27 | 101.85 ± 4.75 | 101.02 ± 2.79 |
| 14 | 118.40 ± 5.10 | 106.75 ± 2.43* | 96.31 ± 2.70* | 101.77 ± 2.25* | 106.56 ± 3.86* | 95.1 ± 3.90* |
| 21 | l29.44 ± 5.39 | 106.54 ± 2.56* | 95.5 ± 2.97* | 101.99 ± 4.00* | 102.l7 ± 3.30* | 91.4 ± 2.48* |

ⁿBaseline
*p < 0.05 compared with vehicle at the same time point

The above description fully discloses how to make and use the present invention. However, this invention is not limited to the particular embodiments described hereinabove, but includes all modification thereof within the scope of the appended claims and their equivalents. Those skilled in the art will recognize through routine experimentation that various changes and modifications may be made without departing from the scope of this invention. The various references to journals, patents and other patent applications that are cited herein are incorporated by reference herein as though fully set forth.

What is claimed is:

1. A method of treating or preventing cardiovascular pathology; comprising, administering a therapeutically effective amount of LXR agonist, or a pharmaceutically acceptable salt, solvate, or physiologically functional derivative thereof.

2. The method of claim 1 in which cardiovascular pathology is selected from the group consisting of cardiac hypertrophy, coronary heart disease, arrhythmia, restricted coronary blood flow, arteriosclerosis, heart failure, congestive heart failure (CHF), and myocardial infarction.

3. A pharmaceutical composition for treating or preventing cardiovascular pathology comprising a therapeutically effective amount of LXR agonist, or a pharmaceutically acceptable salt, solvate, or physiologically functional derivative thereof, and a pharmaceutically acceptable carrier.

4. The pharmaceutical composition of claim 3 in which cardiovascular pathology is selected from the group consisting of cardiac hypertrophy, coronary heart disease, arrhythmia, restricted coronary blood flow, arteriosclerosis, heart failure, congestive heart failure (CHF), and myocardial infarction.

5. The LXR agonist of any one of claims 1 to 4 that is a compound of formula (II): 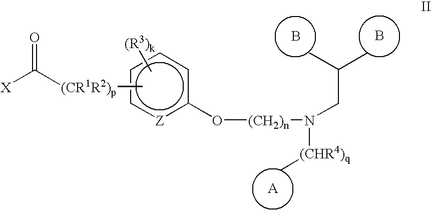
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 04;
when Z is N, k is 0-3;
each R³ is the same or different and is independently selected from the group consisting of halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
a is 0, 1 or 2;
R⁶ is selected from the group consisting of H, C₁₋₈alkyl, C₁₋₈alkoxy and C₂₋₈alkenyl;
each R⁷ and R⁸ are the same or different and are each independently selected from the group consisting of H, C₁₋₈alkyl, C₂₋₈alkenyl, C₃₋₈alkynyl;
R⁹ is selected from the group consisting of H, C₁₋₈alkyl and —NR⁷R⁸;
R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₃₋₈cycloalkyl, aryl, 4-8 membered heterocycle, and 5-6 membered heteroaryl;
each ring B is the same or different and is independently selected from the group consisting of C₃₋₈cycloalkyl and aryl.

6. The LXR agonist of claim 5 that is the compound of formula (IIa)

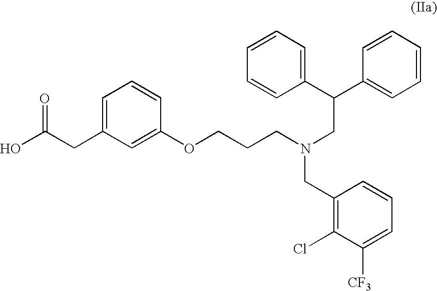

7. The LXR agonist of any one of claims 1 to 4 that is a compound of formula (I): 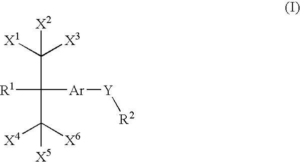
wherein:
Ar represents an aryl group; R¹ is —OH, —O—(C₁-C₇)alkyl, —OC(O)—(C₁-C₇)alkyl, —O—(C₁-C₇)heteroalkyl, —OC(O)— (C₁-C₇)heteroalkyl, —CO₂H, —NH₂, —NH(C₁-C₇)alkyl, —N((C₁-C₇)alkyl)₂ or —NH—S(O)₂—(C₁-C₅)alkyl;
R² is (C₁-C₇)alkyl, (C₁-C₇)heteroalkyl, aryl and aryl(C₁-C₇)alkyl;
X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and
Y is —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— or —N(R¹²)C(O)O—, wherein R12 and R13 are each independently hydrogen, (C₁-C₇)aryl, (C₁-C₇)heteroalkyl, aryl and aryl(C₁-C₇)alkyl, and optionally when Y is —N(R¹²)S(O)ₘ— or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2;
or a pharmaceutically acceptable derivative thereof

8. The LXR agonist of claim 7 that is the compound formula (Ia): [chemical structure]


SEQUENCE LISTING (4 sequences)

SEQ ID NO: 1 - DNA, 1344 bp, Homo sapien
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 2 - protein, 447 aa, Homo sapien
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 3 - DNA, 1383 bp, Homo sapien
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 4 - protein, 460 aa, Homo sapien
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE